(19) United States
(12) Patent Application Publication
(10) Pub. No.: US 2020/0222290 A1
(43) Pub. Date: Jul. 16, 2020

(54) WATER-IN-OIL-TYPE SOLID COSMETIC

(71) Applicant: SHISEIDO COMPANY, LTD., TOKYO (JP)

(72) Inventors: SHUN KUBOTA, Kanagawa (JP); Yukiko SATO, KANAGAWA (JP); Takashi MATSUDA, KANAGAWA (JP); Mayo SHINKE, KANAGAWA (JP); Tomohiro ARAI, KANAGAWA (JP)

(73) Assignee: SHISEIDO COMPANY, LTD., TOKYO (JP)

(21) Appl. No.: 16/630,981

(22) PCT Filed: Jul. 13, 2018
(86) PCT No.: PCT/JP2018/026511

§ 371 Date: Jan. 14, 2020

(30) Foreign Application Priority Data
Jul. 14, 2017 (JP) 2017-137661

(51) Int. Cl. A61K 8/06; A61K 8/02; A61K 8/92; A61Q 17/04

(52) U.S. Cl. CPC A61K 8/064; A61Q 17/04; A61K 8/92; A61K 8/0216

(57) ABSTRACT

The problem to be solved by the present invention is to provide a water-in-oil-type solid cosmetic which contains an ultraviolet ray absorber, can be solidified with a wax, rarely undergoes a sweating phenomenon, and has excellent sensation upon use. A water-in-oil-type solid cosmetic is capable of being solidified with a wax, the solid cosmetic contains a continuous-phase oil that contains an organic modified silicone and can form a continuous oily phase, a wax that is compatible with the continuous-phase oil, an ultraviolet ray absorber and an aqueous phase, wherein oily phase droplets each contain the ultraviolet ray absorber and aqueous phase droplets are dispersed separately in the continuous phase oil having the wax dissolved therein.

### RELATED APPLICATION

This application claims the priority to Japanese Patent Application No. 2017-137661, filed on Jul. 14, 2017, the disclosure of which is incorporated herein by reference in its entirety.

### FIELD OF THE INVENTION

The present invention relates to a water-in-oil solid cosmetic, and particularly to an improvement in the solidification mechanism of the oil-phase thereof.

### BACKGROUND OF THE INVENTION

Water-in-oil solid cosmetics represented by stick-type lipsticks blended with aqueous components are those that are solidified by solidification of a wax in a continuous oil-phase in a mesh-form or formation of a network structure by oily gelling agents.

Water-in-oil solid cosmetics blended with aqueous components, however, had problems in stability over time, wherein not only deformation thereof occurs readily, but also the components comprised in the composition tend readily to soak out to the outer surface of the cosmetic, which is called a perspiration phenomenon. This perspiration phenomenon is remarkable when waxes are used as solidifying agents for the oil-phase. This phenomenon may occur in oily solid cosmetics not blended with water, and the occurrence mechanism thereof still remains unclear.

Patent Literatures 1 to 5 disclose oily solid cosmetics or water-in-oil solid cosmetics that use waxes as the solidifying agents, but it was difficult to achieve providing stability, representatively with regard to such as suppression of perspiration while maintaining excellent feeling in use.

In particular, when ultraviolet light absorbers are blended, it is advantageous in the point that ultraviolet light protecting effect can be provided, but on the other hand, perspiration phenomenon tends to occur readily: thus, development of effective solution therefor has been desired.

### CITATION LIST

### Patent Literatures

PATENT LITERATURE 1: Japanese Unexamined Patent Publication No. H9-20620 A
PATENT LITERATURE 2: Japanese Unexamined Patent Publication No. 2003-63919 A
PATENT LITERATURE 3: Japanese Unexamined Patent Publication No. 2005-314258 A
PATENT LITERATURE 4: Japanese Unexamined Patent Publication No. 2005-314257 A
PATENT LITERATURE 5: Japanese Unexamined Patent Publication No. 2014-129274 A

### DISCLOSURE OF THE INVENTION

### Problem to be Solved by the Invention

The present invention has been made in view of the above-mentioned conventional art, and an object of the present invention is to provide a water-in-oil solid cosmetic that comprises an ultraviolet light absorber, is solidified with a wax, hardly causes perspiration phenomenon, and is excellent in feeling in use.

### Means to Solve the Problem

As stated above, the mechanism of occurrence of a perspiration phenomenon in oily solid cosmetics and water-in-oil solid cosmetics is not sufficiently explained, but it has been considered that the higher the uniformity of the continuous oil-phase excluding the wax is, the better the result is. Accordingly, when ultraviolet light absorbers were to be blended, it has been a general knowledge to devise the formulation of the oil component of the continuous-phase oil such that the ultraviolet light absorber is solidified (at the same time) while dispersing uniformly in the continuous-phase oil.

The present inventors have diligently investigated on the above-mentioned problem, and it is unexpectedly found that a perspiration phenomenon hardly occurs in the oily solid cosmetic in which the wax does not form a network structure upon solidification and an ultraviolet light absorber is separated from the continuous-phase oil as an oil droplet. Moreover, it is found that when an aqueous-phase component is added to prepare a water-in-oil solid cosmetic, an excellent feeling in use (particularly, non-oily feeling) can be achieved even if the ultraviolet light absorber is blended in a large amount; and thereby the present invention is complete.

That is, the present invention comprises the following.

[1] A water-in-oil solid cosmetic comprising:

a continuous-phase oil that comprises an organic modified silicone and forms a continuous oil-phase;

a wax that is compatibly soluble in the continuous-phase oil; and

an ultraviolet light absorber; and

an aqueous-phase,

wherein the water-in-oil solid cosmetic is solidified by the wax; and an oil-phase droplet, which comprises the ultraviolet light absorber, and an aqueous-phase droplet are respectively dispersed in the continuous-phase oil in which the wax is dissolved.

[2] The water-in-oil solid cosmetic according to [1], wherein the aqueous-phase is 5 to 50% by mass in the total weight of the cosmetic.

[3] The water-in-oil solid cosmetic according to [1] or [2], wherein a linear-chain silicone oil is 50% by mass or more in the continuous-phase oil.

[4] The water-in-oil solid cosmetic according to any one of [1] to [3], wherein the ultraviolet light absorber in the continuous oil-phase is 3 to 40% by mass in the total weight of the cosmetic.

[5] The water-in-oil solid cosmetic according to any one of [1] to [4], wherein the oil component comprises: an ester that is a liquid at 25° C. and 1 atmospheric pressure (atm); the organic modified silicone comprising a benzene ring in a chemical structure; and/or a hydrocarbon oil having a boiling point, of 300° C. or lower at 1 atm, and each content of the oil components is respectively 30% by mass or less in the oil component.

[6] A water-in-oil solid cosmetic comprising:

a continuous-phase oil that comprises an organic modified silicone and forms a continuous oil-phase;

the wax that is compatibly soluble in the continuous-phase oil;

an ultraviolet light absorber; and

an aqueous-phase,

wherein the water-in-oil solid cosmetic is solidified by the wax, and the ultraviolet light absorber is 3 to 40% by mass in the total weight of the cosmetic.

[7] The water-in-oil solid cosmetic according to [6], wherein the aqueous-phase is 5 to 50% by mass in the total weight of the cosmetic.

[8] The water-in-oil solid cosmetic according to [6] or [7], wherein a linear-chain silicone oil is 50% by mass or more in the continuous-phase oil.

[9] The water-in-oil solid cosmetic according to any one of [6] to [8], wherein the oil component comprises: an ester that is a liquid at 25° C. and 1 atm; the organic modified silicone having a benzene ring in a chemical structure thereof; and/or a hydrocarbon oil that has a boiling point of 300° C. or lower at 1 atm, and each content of the oil components is respectively 30% by mass or less.

[10] The water-in-oil solid cosmetic according to any one of [6] to [9], wherein an oil-phase droplet, which comprises the ultraviolet light absorber, and an aqueous-phase droplet are respectively dispersed in the continuous-phase oil in which the wax is dissolved.

### Effect of the Invention

A water-in-oil solid cosmetic that perspiration phenomenon hardly occurs and is excellent in feeling use (free of oily feeling) is provided by the present invention.

### BRIEF DESCRIPTION OF THE DRAWINGS

FIG. 1 shows microscopic photographs of the solid oil-phase thin films of Test examples 1-1 to 1-6 (×400).

FIG. 2 shows photographs of the solid oil-phases of Test examples 3-1 to 3-5.

### MODE FOR CARRYING OUT THE INVENTION

Preferable embodiments of the present invention will be described hereinbelow.

### <Continuous-Phase Oil>

A “continuous-phase oil” in the present invention is an oil component that forms a continuous-phase and does not include waxes and ultraviolet light absorbers. The “continuous oil-phase (or may be called simply as “oil-phase”)” in the present invention comprises the continuous-phase oil, an oil component consisting of other oil components, and other oily components (including waxes, ultraviolet light absorbers, powders dispersed in the continuous-phase oil, and the like).

The continuous oil-phase according to the present invention comprises an organic modified silicone oil as an essential component. The silicone-based oil component is not limited in particular as long as the effect of the present invention is not inhibited, and linear polysiloxanes, cyclic polysiloxanes, modified silicones, silicone-based resins, or the like can be used. However, those having a boiling point of 400° C. or lower at normal pressure (i.e. 1 atm) are preferable. Examples thereof include, but not limited to: linear polysiloxanes such as dimethylpolysiloxane, methylphenyl polysiloxane, and methyl hydrogen polysiloxane; and cyclic polysiloxanes such as octamethylcyclotetrasiloxane, decamethylcyclopentasiloxane, dodecamethylcyclohexasiloxane, and tetramethyltetrahydrogencyclotetrasiloxane.

Among them, volatile silicone oils such as: volatile linear polysiloxanes such as a low polymeric dimethylpolysiloxane (degree of polymerization: 3 to 7); or volatile cyclic polysiloxanes such as decamethylcyclopentasiloxane, and octamethylcyclotetrasiloxane are particularly preferable because oily feeling hardly remains when applied to skin, and thus fresh feeling can be achieved.

On the other hand, it is not preferable to blend methylphenylpolysiloxanes in a large amount because an Si/C ratio (ratio between the number of Si atoms and Ci atoms in a molecule) becomes smaller, and thus compatibility with respect to polar oils tends to increase.

In the present invention, one or two or more kinds of the organic modified silicone oils can be used in combination. In the present invention, the blending amount of the organic modified silicone is preferably 50% by mass or more in the continuous-phase oil.

Other oil components can be blended to the oil component of the present invention within the range of not inhibiting the effect of the present invention. The other oil components are not limited in particular as long as they are generally used in cosmetics. Examples thereof include, but not limited to, ester oils, hydrocarbon oils, fatty acids, and higher alcohols. Among the above-mentioned oil components, esters that are liquid at 25° C. and 1 atm, silicone oils that have low Si/C ratios such as organic modified silicones comprising benzene rings in the chemical structure, and hydrocarbon oils that have a boiling point of 300° C. or lower at 1 atm are generally used in cosmetics, and they can be blended alone or in a combination of two or more kinds. These other oil components are preferably 30% by mass or less in the oil component, respectively.

<High Melting Point Wax>

A wax having a high melting point is preferable as the wax to be used in the present invention, and the wax having a solidifying temperature of 70° C. or more, and particularly 75° C. or more is preferable.

The kind of the wax can be selected optionally. When it is used as a mixture, it is preferable that the solidification temperature as the mixture is greater than the above-mentioned temperature.

Examples of waxes that can be used in the present invention include, but not limited to: hydrocarbon-based oils such as polyethylene wax, microcrystalline wax, and paraffin wax; and plant-based waxes such as carnauba wax, rice bran wax, candelilla wax, and ceresin wax. In particular, hydrocarbon-based waxes blended with one or two or more kinds of polyethylene wax, microcrystalline wax, paraffin wax and ceresine wax; carnauba wax; rice bran wax; and candelilla wax are preferred.

The blending amount of the wax is preferably 5 to 30% by mass in the total oil-phase. When it is less than 5% by mass, solidification may be difficult. When it exceeds 30% by mass, it tends to be too hard.

<Ultraviolet Light Absorber>

The ultraviolet light absorber that comprises a benzene ring in the chemical structure is preferably used in the present invention. Examples thereof include, but not limited to: cinnamic acid derivatives such as ethylhexyl methoxycinnamate, isopropyl methoxycinnamate, and isoamyl methoxycinnamate; para-aminobenzoic acid (hereinafter abbreviated as PABA) derivatives such as PABA, ethyl PABA, ethyl-dihydroxypropyl PABA, ethylhexyl-dimethyl PABA, and glyceryl PABA; salicylic acid derivatives such as homosalate, ethylhexyl salicylate, dipropylene glycol salicylate, and TEA salicylate; benzophenone derivatives such as benzophenone-1, benzophenone-2, benzophenone-3 or oxybenzone, benzophenone-4, benzophenone-5, benzophenone-6, benzophenone-8, benzophenone-9, and benzophenone-12; benzylidene camphor derivatives such as 3-benzylidene camphor, 4-methyl-benzylidene camphor, benzylidene camphor sulfonic acid, methosulfate camphor benzalkonium, terephthalylidene dicamphor sulfonic acid, and polyacrylamide methylbenzylidene camphor; triazine derivatives such as anisotriazine, ethylhexyl triazone, diethylhexyl butamidotriazone, 2,4,6-tris(diisobutyl-4′-aminobenzalmalonate)-s-triazine, 2,4-bis-{[4-(2-ethylhexyloxy)-2-hydroxy]-phenyl}-6-(4-methoxyphenyl)-1,3,5-triazine, and 2,4,6-tris[4-(2-ethylhexyloxycarbonyl)anilino]1,3,5-triazine; phenylbenzimidazole derivatives such as phenyldibenzimidazole tetrasulfonic acid disodium salt; phenylbenzotriazole derivatives such as drometrizole, trisiloxane, and methylene bis(benzotriazolyl tetramethylbutylphenol); anthranyl derivatives such as menthyl anthranilate; imidazoline derivatives such as ethylhexyldimethoxybenzylidenedioxoimidazoline propionate; benzalmalonate derivatives such as polyorganosiloxanes having a benzalmalonate functional group; 4,4-diarylbutadiene derivatives such as 1,1-dicarboxy(2,2′-dimethylpropyl)-4,4-diphenylbutadiene; diethylaminohydroxybenzoyl hexyl benzoate; bis-ethylhexyloxyphenol methoxyphenyl triazine; and homosalate.

In terms of feeling in use, the blending amount of the ultraviolet light absorber in the present invention is 3 to 40% by mass, preferably 5 to 35% by mass, and particularly preferably 10 to 30% by mass with respect to the total amount of the oil-phase.

<Aqueous-Phase>

In the present invention, the aqueous-phase can comprise not only water, but also various water-soluble components such as alcohols, water-soluble thickeners, polyhydric alcohols, and water-soluble drugs. The ratio of the aqueous-phase in the cosmetic is preferably 5 to 50% by mass, and particularly preferably 10 to 30% by mass.

As the components that compose the aqueous-phase, components that can be usually used in cosmetics and quasi-drugs can be blended within the range of not inhibiting the stability of the composition. Examples of moisturizers include, but not limited to, 1,3-butylene glycol, polyethylene glycol, propylene glycol, dipropylene glycol, hexylene glycol, glycerin, diglycerin, xylitol, maltitol, maltose, and D-mannitol. Examples of water-soluble polymers include, but not limited to: plant-based polymers such as gum Arabic, carrageenan, pectine, agar, quince seed (cydonia oblonga), starch, and algae colloids (brown algae extracts); microorganism-based polymers such as dextran, and pullulan; animal-based polymers such as collagen, casein, and gelatin; starch-based polymers such as carboxymethyl starch and methylhydroxypropyl starch; alginic acid-based polymers such as sodium alginate; vinyl-based polymers such as carboxyvinyl polymer (CARBOPOL, etc.); polyethylene-based polymers; polyoxyethylene polyoxypropylene copolymer polymers; acrylic polymers such as sodium polyacrylate and polyacrylamide; and inorganic water-soluble polymers such as bentonite, magnesium aluminum silicate and laponite.

As water-soluble polymers, homopolymers or copolymers of 2-acrylamide-2-methylpropane sulfonic acid (hereinafter abbreviated as “AMPS”) can be used. The copolymer is a copolymer consisting of comonomers such as vinylpyrrolidone, amide acrylate, sodium acrylate, hydroxyethyl acrylate, and the like. That is, AMPS homopolymer, vinylpyrrolidone/AMPS copolymer, dimethyl acrylamide/AMPS copolymer, amide acrylate/AMPS copolymer, sodium acylate/AMPS copolymer, and the like can be used, too.

Other than the above-mentioned components, other components that are usually used in cosmetics can be blended to the cosmetic of the present invention within the scope of not inhibiting the object and effect of the present invention, as appropriate and as necessary. Examples of such components include, but not limited to, water-soluble polymers other than those listed above, oil-soluble polymers, polymer powders, emulsifiers, alcohols, ester oils, hydrocarbon oils, fatty acids, higher alcohols, drugs, ultraviolet light scattering agents, organic modified clay minerals, dyes, pigments, perfumes, and film forming agents.

<Surfactants>

Since the present invention is also a water-in-oil emulsion, silicone surfactants can be blended as the emulsifiers thereof. Silicone surfactants are not limited in particular as long as they can be used in a system of water-in-oil emulsions. Examples thereof include, but not limited to, poly(oxyethylene/oxypropylene) methylpolysiloxane copolymer, polyoxyethylene methylpolysiloxane copolymer, silicone-chain branched methylpolysiloxane copolymer, alkyl-chain branched polyoxyethylene methylpolysiloxane copolymer, alkyl-chain/silicone-chain branched polyoxyethylene methylpolysiloxane copolymer, crosslinked polyoxyethylene methylpolysiloxane, alkyl-group containing crosslinked polyoxyethylene methylpolysiloxane, branched polyglycerin modified silicone, crosslinked polyglycerin modified silicone, alkyl-group containing crosslinked polyglycerin modified silicone, and alkyl-group branched polyglycerin modified silicone.

Examples of poly(oxyethylene/oxypropylene) methylpolysiloxane copolymers include, but not limited to: PEG/PPG-20/22 butyl ether dimethicone (“KF-6012”, manufactured by Shin-Etsu Chemical Co., Ltd.); PEG/PPG-20/20 dimethicone (“BY22-008M”, manufactured by Dow Corning Toray Silicone Co., Ltd.); lauryl PEG/PPG-18 methicone (“5200 Formulation Aid”, manufactured by Dow Corning Toray Co., Ltd.); PEG/PPG-19/19 dimethicone (“5330 Fluid”, manufactured by Dow Corning Toray Co., Ltd.); and PEG/PPG-15/I5 dimethicone (“5330 Fluid”, manufactured by Dow Corning Toray Co., Ltd.).

Examples of polyoxyethylene methylpolysiloxane copolymers include, but not limited to: PEG-11 methyl ether dimethicone (“KF-6011”, manufactured by Shin-Etsu Chemical Co., Ltd.); PEG-9 dimethicone (“KF-6013”, manufactured by Shin-Etsu Chemical Co., Ltd.); PEG-3 dimethicone (“KF-6015”, manufactured by Shin-Etsu Chemical Co., Ltd.); PEG-9 methyl ether dimethicone (“KF-6016”, manufactured by Shin-Etsu Chemical Co., Ltd.); PEG-10 dimethicone (“KF-6017”, manufactured by Shin-Etsu Chemical Co., Ltd.); PEG-11 methyl ether dimethicone (“KF-6018”, manufactured by Shin-Etsu Chemical Co., Ltd.); PEG-9 dimethicone (“KF-6019”, manufactured by Shin-Etsu Chemical Co., Ltd.); and PEG-12 dimethicone (“SH3771M”, “SH3772M”, “SH3773M”, “SH3775M”, and the like, manufactured by Dow Corning Toray Co., Ltd.).

When the surfactant is used in a large amount, the ultraviolet light absorber (polar oil) may be dissolved in the continuous-phase oil. Therefore, the blending amount of the surfactant in the continuous-phase oil is 5% by mass or less, preferably 2.5% by mass or less, more preferably 1% by mass or less, and most preferably 0.5% by mass or less.

Examples of oil-soluble polymers include, but not limited to, trimethylsiloxysilicate, alkyl modified silicone, and polyamide modified silicone.

Examples of polymer powders include, but not limited to, dimethicone crosspolymer, dimethicone/vinyl dimethicone crosspolymer, polymethylsilsesquioxane, polyethylene, and polymethylmethacrylate.

Examples of emulsifiers other than the above include, but not limited to, fatty acid esters such as glycerin fatty acid ester, polyglycerin fatty acid ester, polyoxyethylene glycerin fatty acid ester, sorbitan fatty acid ester, and polyoxyethylene sorbitan fatty acid ester.

The water-in-oil solid cosmetic of the present invention can be used preferably as skin-care cosmetics, makeup cosmetics, and hair cosmetics. In particular, uses as makeup cosmetics such as: foundations; makeup bases; color controllers; concealers; eye colors; lipsticks; and body creams, and sunscreen cosmetics are preferable. Uses as foundations, sunscreens and lipsticks are particularly preferable.

<Production Method>

The solid cosmetic of the present invention can be produced by a method comprising steps of: heating and mixing an oil-phase component, and mixing a powder thereto; and separately mixing another oil-phase component comprising a wax and an ultraviolet light absorber by heating when needed, and emulsifying a pre-heated aqueous-phase component in the obtained oil-phase component.

An example of the specific procedure is as follows; a mixture of the oil-phase component comprising a wax is heated to 80 to 90° C. and dissolved, and an ultraviolet light absorber, an emulsifier is optionally added thereto, and the mixture is further stirred using a homomixer to make an emulsion. While the emulsion is in a fluid state, the emulsion is filled into a container, and then it is cooled and solidified to obtain the solid cosmetic of the present invention.

### Examples

The present invention will be described with reference to the following examples. However, the present invention is not limited to the following examples. First of all, the methods used in the examples will be described. The numerical values in Tables 1 to 4 show “part by weight”, and the numerical values in Tables 5 and 6 show “% by mass” unless otherwise specified. Moreover, the blanks in the tables show that the blending amount is “0”.

<Production Method of Solid Oil-Phase>

A wax heated to 80° C. and dissolved was added to a mixture of the oil-phase components comprising the ultraviolet light absorber. The resulting mixture was heated to 80 to 90° C. and dissolved homogeneously. The resulting oil-phase was applied onto a slide glass, and solidified at room temperature to obtain a thin film formed of the solid oil-phase.

<Evaluation Method>

### Perspiration

A sample stored for two weeks at 50° C. was taken out at a condition of 25° C. and was allowed to be cooled. Then, it was confirmed whether oil droplets can be observed on the surface of the sample.
No: No oil droplet exists.
Yes: Oil droplets exist.

### Hardness

Hardness of the solid oil-phase was measured by Sun Rheo Meter (CMPAC11, manufactured by Sun Scientific Co., Ltd.) with 5.6 mm φ disk needle at a penetration of 1 mm and a penetration speed of 20 mm/min, and by peak reading. The rheometer hardness of the solid cosmetic is desired to be 50 or more and 300 or less, and preferably 60 or more and 250 or less. When the rheometer hardness is 50 or less, the solidification force of the oil-phase becomes weak, and perspiration easily occurs. Moreover, the shock-resistance of the content becomes weak, and the content may easily become uneven during transportation or the like. On the other hand, when the rheometer hardness is 300 or more, it becomes difficult to take out the content, and usability may be poor, too.

### Inner Structure of Solid Oil-Phase

The thin film formed of the solid oil-phase was observed with a microscope. Existence of a network structure formed of the solidified wax, uniformity of the parts other than the network structure, and existence of oil droplets were evaluated.

### Compatibility

The oil-phase comprising the wax was heated to determine whether it is a transparent single-phase under a condition of 80 to 90° C. In the present application, A and B was determined as being compatible, and C was determined as not being compatible.

A: It was a transparent single-phase under a condition of 80 to 90° C.
B: It was semi-transparent under a condition of 80 to 90° C.
C: It was clouded and opaque under a condition of 80 to 90° C.

### Feeling in Use

10 trained-expert panelists applied the sample to evaluate feeling in use in accordance with the following criteria. In the present application, A and B was determined as not being oily, and C was determined as being oily.

A: 7 or more and 10 or less panelists answered that there was no oily feeling.
B: 4 or more and 6 or less panelists answered that there was no oily feeling.
C: 3 or less panelists answered that there was no oily feeling.

<Production Method of Water-in-Oil Solid Cosmetic>

To a mixture of the oil-phase components comprising the wax, which was heated to 80 to 90° C. and dissolved, and the ultraviolet light absorber, an emulsifier was added thereto, and the resulting mixture was stirred using a homomixer to give an emulsion. Then, while the emulsion is in a fluid state, it was filled into a container, and was cooled and solidified to obtain a solid cosmetic.

Detailed information of the components shown with asterisks in the tables is as follows.

*1: PARSOL® 1789 (manufactured by DSM Nutrition Japan K.K.)
*2: A mixed solution prepared at a ratio of: 5 parts by weight of octyl methoxycinnamate; 3 parts by weight of octocrylene; and 2.5 parts by weight of 4-tert-butyl-4′-methoxydibenzoylmethane.
*3: SILICONE SC9450N (manufactured by Shin-Etsu Chemical Co., Ltd.)
*4: GANZPEARL® GMX-0810 (manufactured by Ganz Chemical Co., Ltd.)

### Example 1: Oil Component and Perspiration Phenomenon

First of all, the present inventors investigated on the oil component which the wax and the ultraviolet light absorber are dissolved. The solid oil-phases having the formulations shown in Table 1 were produced in accordance with the above-mentioned method and were evaluated for the items listed in the table. The results are shown in Table 1.

TABLE 1

| Test Examples | | | 1-1 | 1-2 | 1-3 | 1-4 | 1-5 | 1-6 |
|---|---|---|---|---|---|---|---|---|
| Formu- | Oil | Decamethylcyclopentasiloxane | 14.72 | | | | | |
| lation | com- | Polydimethylsiloxane(2CS) | | 14.72 | | | | |
| | ponent | Dodecamethyl- | 10 | 10 | 10 | 10 | 10 | 10 |
| | | cyclohexasiloxane | | | | | | |
| | | Macadamia nut oil | | | 14.72 | | | |
| | | Jojoba oil | | | | 14.72 | | |
| | | Diphenylsiloxy | | | | | 14.72 | |
| | | phenyl trimethicone | | | | | | |
| | | Light liquid paraffin | | | | | | 14.72 |
| | Ultra- | Octyl methoxycinnamate | 5 | 5 | 5 | 5 | 5 | 5 |
| | violet | Octocrylene | 3 | 3 | 3 | 3 | 3 | 3 |
| | light | 4-tert-butyl-4′-methoxy | 2.5 | 2.5 | 2.5 | 2.5 | 2.5 | 2.5 |
| | absorber | dibenzoylmethane *1 | | | | | | |
| | Wax | Paraffin wax | 3.9 | 3.9 | 3.9 | 3.9 | 3.9 | 3.9 |
| | | Hydrogenated jojoba oil | 4.6 | 4.6 | 4.6 | 4.6 | 4.6 | 4.6 |
| Eval- | Perspiration | | No | No | Yes | Yes | Yes | Yes |
| uatio | Hardness (1.5 φ rheometer) | | 99 | 94 | 80 | 80 | 112 | 74 |
| | Inner | Network structure formed | No | No | Yes | Yes | Yes | Yes |
| | structure | of solidified wax | | | | | | |
| | | Oil droplets | Yes | Yes | No | No | No | No |

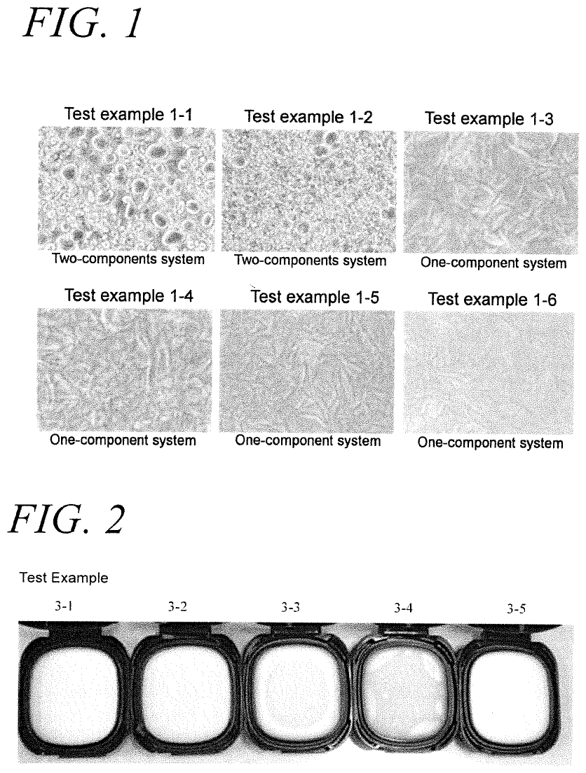

As a result of microscope observation, in the solid oil-phase having macadamia nut oil or jojoba oil (both of them having fatty acid as the main component) as the main oil component and dodecamethylcyclohaxasiloxane (boiling point under normal pressure: 245° C.) as the sub-oil-component, a network structure formed of the solidified wax was observed over the whole area of the solid phase, and voids thereof were uniform (Test examples 1-3, 1-4, Table 1 and FIG. 1). It was similar when diphenylsiloxy phenyl trimethicone (boiling point under normal pressure: 260° C. or more, Test example 1-5) or a light liquid paraffin (hydrocarbon oil) was used as the main oil component (Table 1 and FIG. 1). Accordingly, in these Test examples (1-3 to 1-6), it is considered that the wax is deposited in form of a network upon solidification, and other components (i.e. the ultraviolet light absorber and the oil component) are solidified in a uniformly dissolved state. In these solid oil-phases, perspiration phenomenon was observed (Test examples 1-3 to 1-6, Table 1 and FIG. 1).

On the other hand, when decamethylcyclohexasiloxane (boiling point under normal pressure: 210° C.) or dimethylpolysiloxane (2CS; boiling point under normal pressure: 229° C.) was used as the main oil component, the network structure like in the above-mentioned test examples was not observed, and numerous oil droplets were observed in the uniform solid phase (Test examples 1-1, 1-2, Table 1 and FIG. 1). In these solid oil phases, perspiration phenomenon was not observed (Test examples 1-1, 1-2).

Accordingly, it became apparent that even if the same ultraviolet light absorber and wax component were used, the structure of the solid oil phase obtained after solidification changes dramatically when the oil component that dissolves the ultraviolet light absorber and wax is changed. Moreover, since the test examples in which perspiration phenomenon was observed (Test examples 1-3 to 1-6) and the test examples in which perspiration phenomenon was not observed (Test examples 1-1, 1-2) had common characteristics respectively, it was strongly indicated that there is a structure of the solid oil-phase which hardly causes perspiration phenomenon.

Furthermore, it became apparent that the organic modified silicone oil is preferable as the oil component which hardly causes perspiration phenomenon.

### Example 2: Investigation on Compatibility

In the test examples that perspiration phenomenon was not observed in Example 1 (Test examples 1-1, 1-2), they were in common in the structure of the wax, and also in the point that oil droplets were generated. Therefore, in order to understand the reason why these phenomenon occurred in these test examples, compatibility of the oil component and each component was investigated.

First of all, compatibility of polydimethylsiloxane used as the main oil component in Test example 1-2 and the ultraviolet light absorber was analyzed. Polydimethylsiloxane (2CS) having a low molecular weight (i.e. low viscosity) was used in Test example 1-2, but polydimethylsiloxanes having higher molecular weights were also analyzed. The results are shown in Table 2.

TABLE 2

| Test Examples | | | 2-1 | 2-2 | 2-3 | 2-4 | 2-5 |
|---|---|---|---|---|---|---|---|
| Formu- | Oil | Polydimethylsiloxane (2CS) | 30 | | | | 25 |
| lation | com- | Polydimethylsiloxane (6CS) | | 30 | | | |
| | ponent | Polydimethylsiloxane (20CS) | | | 30 | | |
| | | Polydimethylsiloxane (100CS) | | | | 30 | 5 |
| | Ultra- | Octyl methoxycinnamate | 5 | 5 | 5 | 5 | 5 |
| | violet | Octocrylene | 3 | 3 | 3 | 3 | 3 |
| | light | 4-tert-butyl-4′-methoxydibenzoylmethane *1 | 2.5 | 2.5 | 2.5 | 2.5 | 2.5 |
| | absorber | | | | | | |

| | | | | | | |
|---|---|---|---|---|---|---|
| Eval- | Compatibility | No | No | No | No | No |
| uation | | | | | | |

It became apparent that octyl methoxycinnamate, octocrylene, and 4-tert-butyl-4′-methoxydibenzoylmethane, which are ultraviolet light absorbers, are not compatible with any of dimethylpolysiloxanes having viscosities of 2CS to 100CS. It is generally known that ultraviolet light absorbers are oily components that have a high polarity and is difficult to be compatible with oil components that have a low polarity (e.g. silicone oil).

Next, compatibility with wax was analyzed. The results are shown in Table 3.

TABLE 3

| Test Examples | | | 3-1 | 3-2 | 3-3 | 3-4 | 3-5 | 3-6 | 3-7 |
|---|---|---|---|---|---|---|---|---|---|
| Formu- | Oil | Polydimethylsiloxane (2CS) | 30 | | | | 25 | | |
| lation | com- | Polydimethylsiloxane (6CS) | | 30 | | | | | |
| | ponent | Polydimethylsiloxane (20CS) | | | | 30 | | | |
| | | Polydimethylsiloxane (100CS) | | | | | 30 | 5 | |
| | Ultraviolet | Octyl methoxycinnamate | | | | | | 30 | |
| | light | Octocrylene | | | | | | | 30 |
| | absorber | | | | | | | | |
| | Wax | Paraffin wax | 3.9 | 3.9 | 3.9 | 3.9 | 3.9 | 3.9 | 3.9 |
| | | Hydrogenated jojoba oil | 4.6 | 4.6 | 4.6 | 4.6 | 4.6 | 4.6 | 4.6 |

| | | | | | | | | |
|---|---|---|---|---|---|---|---|---|
| Eval- | Hardness (1.5 φ) | 98 | 73 | ≥200 | ≥200 | 82 | liquid | liquid |
| uation | Compatibility | A | A | B | B | A | A | A |

The wax was compatible with dimethylpolysiloxane having low molecular weights (2CS, 6CS) (Test examples 3-1, 3-2, Table 3 and FIG. 2), but was not sufficiently compatible with dimethylpolysiloxane having high molecular weights (20CS, 100CS) (Test examples 3-3, 3-4, Table 3 and FIG. 2). However, even if dimethylpolysiloxane having a high molecular weight (100CS) was used, it became compatible when it was mixed with dimethylpolysiloxane having a low molecular weight (2CS) (Test example 3-5, Table 3 and FIG. 2). It is considered that since the weight of the terminal methyl group increases relatively when polydimethylsiloxane having a low molecular weight is used, the Si/C ratio decreases and thus compatibility of the wax increases.

Interestingly, when the ultraviolet light absorber and the wax were mixed, the wax did not solidify and remained as a liquid (Test examples 3-6, 3-7, Table 3)

From the results of Tables 2 and 3, among the components of Test example 1-2, it was indicated that the wax component is compatible with dimethylpolysiloxane (2CS), which is the main oil component, but the ultraviolet light absorber is not compatible with the main oil component. Furthermore, the results were similar in dodecamethylcyclohexasiloxane, which is the sub-oil-component (results not disclosed).

Therefore, in the test examples in which the perspiration phenomenon was not observed, it became apparent that the wax component is solidified while it is dissolved in the oil-phase, but the ultraviolet light absorber component is rejected from the oil component along with solidification and changes into oil droplets.

### Example 3: Investigation on Ratio of Organic Modified Silicone Oil in Oil Component

Investigation was carried out for the oil component formulation that can form the structure of the wax and change the ultraviolet light absorber into oil droplets upon solidification. Polydimethylsiloxane (6CS), which was confirmed to have these properties, was mixed with oil components, which do not (or are considered not to) have these properties, and the inner structure and the perspiration phenomenon of the solid oil-phase to be obtained were analyzed.

The results are shown in Table 4.

TABLE 4

| Test Examples | | | 4-1 | 4-2 | 4-3 | 4-4 | 4-5 | 4-6 | 4-7 | 4-8 | 4-9 | 4-10 |
|---|---|---|---|---|---|---|---|---|---|---|---|---|
| Formu- | Oil | Polydimethylsiloxane(6CS) | 0 | 10 | 20 | 30 | 40 | 50 | 60 | 20 | 20 | 20 |
| lation | com- | Diphenylsiloxy phenyl | 60 | 50 | 40 | 30 | 20 | 10 | 0 | | | |
| | ponent | trimethicone | | | | | | | | | | |
| | | Isopropyl myristate | | | | | | | | 40 | | |
| | | Glyceryl | | | | | | | | | 40 | |
| | | tris(2-ethylhexanoate) | | | | | | | | | | |
| | | Isododecane | | | | | | | | | | 40 |
| | Ultra- | Octyl methoxycinnamate | 10 | 10 | 10 | 10 | 10 | 10 | 10 | 10 | 10 | 10 |
| | violet | Octocrylene | 6 | 6 | 6 | 6 | 6 | 6 | 6 | 6 | 6 | 6 |
| | light | 4-tert-butyl-4′- | 4 | 4 | 4 | 4 | 4 | 4 | 4 | 4 | 4 | 4 |
| | absorber | methoxydibenzoyl- | | | | | | | | | | |
| | | methane *1 | | | | | | | | | | |
| | Wax | Paraffin wax | 10 | 10 | 10 | 10 | 10 | 10 | 10 | 10 | 10 | 10 |
| | | Hydrogenated jojoba oil | 10 | 10 | 10 | 10 | 10 | 10 | 10 | 10 | 10 | 10 |
| Eval- | Ratio of polydimethylsiloxane | | 0.0 | 16.7 | 33.3 | 50.0 | 66.7 | 83.3 | 100.0 | 33.3 | 33.3 | 33.3 |
| uation | in the oil component (%) | | | | | | | | | | | |
| | Perspiration | | Yes | Yes | Yes | No | No | No | No | Yes | Yes | Yes |
| | Inner | Network structure formed | Yes | Yes | Yes | No | No | No | No | Yes | Yes | Yes |
| | structure | of solidified wax | | | | | | | | | | |
| | | Oil droplets | No | No | No | Yes | Yes | Yes | Yes | No | No | No |

When polydimethylsiloxane (6CS) and diphenylsiloxy phenyl trimethicone used in Test example 1-2 were used in combination as the oil component, the wax component was deposited, oil droplets were not formed, and the perspiration phenomenon was observed when the ratio of polydimethylsiloxane in the total oil component was 33.3% by mass or less (Test examples 4-1 to 4-3); but the structure formed of the wax component was not observed, oil droplets were formed, and the perspiration phenomenon was not observed when the ratio of polydimethylsiloxane was 50.0 to 100.0% by mass (Test examples 4-4 to 4-6). Moreover, when the ester oils having a high polarity (isopropyl myristate, and glyceryl tris(2-ethylhexanoate)) were blended at 66.7% by mass in the oil component, the wax component was deposited (only the wax structure was formed), oil droplets were not formed, and the perspiration phenomenon was observed (Test examples 4-8, 4-9). Furthermore, when the hydrocarbon oil (isododecane), which is a low polar oil, was blended at 66.7% by mass in the oil component, the wax component was deposited (only the wax structure was formed), oil droplets were not formed (only the wax structure was formed), and the perspiration phenomenon was observed (Test examples 4-8, 4-9).

Accordingly, in order to form the wax structure and change the ultraviolet light absorber into oil droplets upon solidification, it was indicated that the organic modified silicone oil needs to be comprised at 50% by mass or more in the oil component, and polydimethylsiloxane (linear silicone oil) needs to be 50% by mass or more in the organic modified silicone oil. Furthermore, it was indicated that other oil components can be blended at 50% by mass or less in the oil component.

### Example 4: Investigation on Blending Amount of Ultraviolet Light Absorber

From the results of Examples 1 to 3, it became apparent that the perspiration phenomenon hardly occurs in the solid oil-phase that maintains the wax structure and the ultraviolet light absorber as oil droplets; however, it is concerned that oily feeling may be caused upon use by comprising oil droplets. Therefore, the solid oil-phases comprising the ultraviolet light absorber at various ratios were produced to analyze the occurrence of oily feelings. The results are shown in Table 5.

TABLE 5

| Test Examples | | | 5-1 | 5-2 | 5-3 | 5-4 | 5-5 | 5-6 | 5-7 |
|---|---|---|---|---|---|---|---|---|---|
| Formulation | Oil | Polydimethylsiloxane (6CS) | 60 | 60 | 60 | 60 | 60 | 50 | 40 |
| | com- | Dodecamethyl- | 20 | 17 | 15 | 10 | 0 | 0 | 0 |
| | ponent | cyclohexasiloxane | | | | | | | |
| | Ultraviolet light absorber *2 | | 0 | 3 | 5 | 10 | 20 | 30 | 40 |
| | Wax | Paraffin wax | 10 | 10 | 10 | 10 | 10 | 10 | 10 |
| | | Hydrogenated | 10 | 10 | 10 | 10 | 10 | 10 | 10 |
| | | jojoba oil | | | | | | | |
| Evaluation | Perspiration | | No | No | No | No | No | No | No |
| | Inner | Network structure | No | No | No | No | No | No | No |
| | structure | formed of | | | | | | | |
| | | solidified wax | | | | | | | |
| | | Oil droplets | No | Yes | Yes | Yes | Yes | Yes | Yes |

| | | | | | | | |
|---|---|---|---|---|---|---|---|
| Feeling in use | B | B | B | B | B | C | C |
| (non-oily feeling) | | | | | | | |

In the solid oil-phase, which does not comprise the ultraviolet light absorber, the wax was not deposited, and oil droplets were not formed (Test example 5-1). Consequently, it was confirmed that the oil droplets observed in the previous text examples were oil droplets that were formed from the oil-phase having the ultraviolet light absorber as the main component. The perspiration phenomenon was not observed in this solid oil-phase.

In the solid oil-phases comprising the ultraviolet light absorber at 3 to 40% by mass (Test examples 5-2 to 5-7), the wax was not deposited, oil droplets were formed, and the perspiration phenomenon was not observed. Among them, in those blended with 20% by mass or less of the ultraviolet absorber (Test examples 5-2 to 5-5), feeling in use (non-oily feeling) was the same level as one that does not comprise the ultraviolet light absorber (Test example 5-1). On the contrary, those that comprise the ultraviolet light absorber at 30 to 40% by mass (Test examples 5-6, 5-7) had oily feeling and were poor in feeling in use.

Although the ultraviolet light absorber does not promote the perspiration phenomenon when it is blended in a large amount, it was found that the ultraviolet light absorber has a preferable range of the blending amount in terms of feeling in use (non-oily feeling). Moreover, in a state of the solid oil-phase (i.e. no aqueous-phase comprised), it became apparent that significant oily feeling is not felt when the ratio of the ultraviolet light absorber in the total oil-phase is less than 30% by mass.

### Example 5: Production Example of Water-in-Oil Solid Cosmetic

Next, the aqueous-phase component was blended to prepare a water-in-oil emulsion cosmetic, and perspiration phenomenon, the inner structure, and feeling in use were analyzed. Formulations and analysis results are shown in Tables 6 and 7.

TABLE 6

| Test Examples | | | | 6-1 | 6-2 | 6-3 | 6-4 | 6-5 |
|---|---|---|---|---|---|---|---|---|
| Formu- | Oil- | Continuous- | Dodecamethyl- | 14.35 | 11.85 | 9.35 | 6.85 | 4.35 |
| lation | phase | phase oil | cyclohexasiloxane | | | | | |
| | | | Polydimethyl- | 14.72 | 12.22 | 9.72 | 7.22 | 4.72 |
| | | | siloxane (6CS) | | | | | |
| | | Ultraviolet | Octyl methoxycinnamate | 5 | 10 | 15 | 20 | 25 |
| | | light | Octocrylene | 3 | 6 | 9 | 12 | 15 |
| | | absorber | 4-tert-butyl-4′- | 2.5 | 4 | 6 | 8 | 10 |
| | | | methoxydibenzoyl- | | | | | |
| | | | methane *1 | | | | | |
| | | Wax | Paraffin wax | 3.9 | 3.9 | 3.9 | 3.9 | 3.9 |
| | | | Hydrogenated jojoba oil | 4.6 | 4.6 | 4.6 | 4.6 | 4.6 |
| | | Others | Sorbitan sesquiisostearate | 2 | 2 | 2 | 2 | 2 |
| | | | Polyoxyethylene/ | 2 | 2 | 2 | 2 | 2 |
| | | | methylpolysiloxane | | | | | |
| | | | copolymer *3 | | | | | |
| | | | Polymethyl | 5 | 5 | 5 | 5 | 5 |
| | | | methacrylate *4 | | | | | |
| | Aqueous-phase | | Glycerin | 4 | 4 | 4 | 4 | 4 |
| | | | Dipropylene glycol | 5 | 5 | 5 | 5 | 5 |
| | | | EDTA-3NaH₂O | 0.05 | 0.05 | 0.05 | 0.05 | 0.05 |
| | | | Phenoxyethanol | 0.5 | 0.5 | 0.5 | 0.5 | 0.5 |
| | | | Ion-exchanged water | Balance | Balance | Balance | Balance | Balance |

| | Total | | 100.0 | 100.0 | 100.0 | 100.0 | 100.0 |
|---|---|---|---|---|---|---|---|
| Eval- | Ratio of ultraviolet light | | 18.4 | 32.5 | 45.1 | 55.9 | 65.3 |
| | absorber in oil-phase (%) | | | | | | |
| uation | Perspiration | | No | No | No | No | No |
| | Inner | Network structure formed | No | No | No | No | No |
| | structure | of solidified wax | | | | | |
| | | Oil droplets | Yes | Yes | Yes | Yes | Yes |
| | | Emulsified particles | Yes | Yes | Yes | Yes | Yes |

| | | | | | |
|---|---|---|---|---|---|
| Feeling in use (non-oily feeling) | A | A | A | B | C |

TABLE 7

| Test Examples | | | | | 6-6 | 6-7 | 6-8 |
|---|---|---|---|---|---|---|---|
| Formu- | Oil- | Oil | Continuous- | Dodecamethyl- | 11.85 | 10.85 | 10.85 |
| lation | phase | com- | phase oil | cyclohexasiloxane | | | |
| | | ponent | Other oil | Polydimethylsiloxane (6CS) | 12.22 | 11.22 | 11.22 |
| | | | component | Diisopropyl sebacate | 10 | 10 | 10 |
| | | Ultraviolet | | Octyl methoxycinnamate | 5 | 5 | 5 |
| | | light | | Octocrylene | 2 | 2 | 2 |
| | | absorber | | 2,4-bis-{[4-(2-ethylhexyloxy)- | 2 | 2 | 2 |
| | | | | 2-hydroxy]- | | | |
| | | | | phenyl}-6-(4-methoxy- | | | |
| | | | | phenyl)-1,3,5-triazine | | | |
| | | | | 2,4,6-tris[4-(2-ethylhexyloxy- | 1 | 1 | 1 |
| | | | | carbonyl)anilino] | | | |
| | | | | 1,3,5-triazine | | | |
| | | | | Oxybenzone | | 2 | |
| | | | | Diethylaminohydroxy- | | | 2 |
| | | | | benzoyl hexyl benzoate | | | |
| | | Wax | | Paraffin wax | 3.9 | 3.9 | 3.9 |
| | | | | Hydrogenated jojoba oil | 4.6 | 4.6 | 4.6 |
| | | Others | | Sorbitan sesquiisostearate | 2 | 2 | 2 |
| | | | | Polyoxyethylene/ | 2 | 2 | 2 |
| | | | | methylpolysiloxane | | | |
| | | | | copolymer *3 | | | |
| | | | | Polymethyl methacrylate *4 | 5 | 5 | 5 |
| | Aqueous-phase | | | Glycerin | 4 | 4 | 4 |
| | | | | Dipropylene glycol | 5 | 5 | 5 |
| | | | | EDTA-3NaH₂O | 0.05 | 0.05 | 0.05 |
| | | | | Phenoxyethanol | 0.5 | 0.5 | 0.5 |
| | | | | Ion-exchanged water | Balance | Balance | Balance |

| | Total | | | 100.0 | 100.0 | 100.0 |
|---|---|---|---|---|---|---|
| Eval- | | Ratio of ultraviolet light absorber in oil-phase (%) | | 18.4 | 32.5 | 65.3 |
| uation | | Perspiration | | No | No | No |
| | | Inner | Network structure formed of solidified wax | No | No | No |
| | | structure | Oil droplets | Yes | Yes | Yes |
| | | | Emulsified particles | Yes | Yes | Yes |

| | | | |
|---|---|---|---|
| Feeling in use (non-oily feeling) | B | B | B |

The perspiration phenomenon was not observed in any of the cosmetics; and the wax structure was not formed, and oil droplets and emulsified particles were independently present in the uniform solid oil-phase (Test examples 6-1 to 6-5). Feeling in use (non-oily feeling) was significantly excellent in Test examples 6-1 to 6-3 in which the ultraviolet light absorber was blended at 45.1% by mass or less in the oil-phase (the oil-phase as used here includes the continuous-phase oil, the ultraviolet light absorber, the wax and the powder dispersed in the oil-phase). Moreover, remarkable oily feeling was not felt in Test example 6-4 in which the ultraviolet light absorber is comprised at 55.9% by mass in the oil-phase. However, feeling in use was rather oily in Test example 6-4 in which the ultraviolet light absorber is comprised at 55.9% by mass in the oil-phase. It is considered that this change was caused because dewy feeling was provided by comprising emulsified particles consisting of the aqueous-phase components (in addition to oil droplets comprising the ultraviolet light absorber) in the oil-phase.

In Test examples 6-6 to 6-8 in which a large amount of slightly-soluble ultraviolet light absorbers were dissolved by using the ester oil component having a high polarity like the ultraviolet light absorber as the solvent, the perspiration phenomenon was not observed. The structure was not formed, and oil droplets and emulsified particles were independently present in the uniform solid oil-phase (Test examples 6-6 to 6-8), and remarkable oily feeling was not felt.

Thus, it became apparent that the water-in-oil solid cosmetic of which oily feeling is not felt and is excellent in feeling in use can be obtained by blending the ultraviolet light absorber at 3% to 40% in the total weight of the cosmetic, and 5.0 to 56.0% by mass, preferably 10.0 to 55.0% by mass, and more preferably 15.0 to 50.0% by mass as the ratio in the oil-phase.

From the above results, it became apparent that the water-in-oil solid cosmetic which hardly causes the perspiration phenomenon and is excellent in feeling in use can be obtained by blending and emulsifying the following components: the continuous-phase oil formed of the oil component that comprises 50% by mass or more of the organic modified silicone oil and the ratio of polydimethylsiloxane (linear silicone oil) in the silicone oil is 50% by mass or more; the wax that solidifies while it is dissolved in the continuous-phase oil; the ultraviolet light absorber that separates as oil droplets upon solidification; and the aqueous-phase component.

Examples of the present invention are listed in the following, but the present invention is not limited thereto.

### Example 1: Foundation

| | |
|---|---|
| Dodecamethylcylohexasiloxane | 13% |
| Polydimethylsiloxane (6CS) | 13% |
| Octyl methoxycinnamate | 5% |
| Octocrylene | 3% |
| 4-tert-butyl-4′-methoxydibenzoylmethane | 2.5% |
| Paraffin wax | 3.9% |
| Hydrogenated jojoba oil | 4.6% |
| Sorbitan sesquiisostearate | 2% |
| Polyoxyethylene/methylpolysiloxane copolymer | 2% |
| Polymethyl methacrylate | 5% |
| Glycerin | 2% |
| Dipropylene glycol | 6% |
| Phenoxyethanol | 0.5% |
| Hydrophobized pigment-grade titanium oxide | 6% |
| Hydrophobized yellow iron oxide | 1.2% |
| Hydrophobized red iron oxide | 0.4% |
| Hydrophobized black iron oxide | 0.1% |
| Ion-exchanged water | Balance |

### Example 2: Stick-Type Foundation

| | |
|---|---|
| Dodecamethylcylohexasiloxane | 12% |
| Polydimethylsiloxane (6CS) | 12.5% |
| Octyl methoxycinnamate | 7.5% |
| Ethylhexyl triazone | 0.15% |
| Bis-ethylhexyloxyphenol | 0.15% |
| methoxyphenyl triazine | |
| 4-tert-butyl-4′-methoxy- | 0.5% |
| dibenzoylmethane | |
| Paraffin wax | 0.5% |
| Rice bran wax | 2% |
| Sorbitan sesquiisostearate | 2% |
| Polyoxyethylene/methyl- | 2% |
| polysiloxane copolymer | |
| Polymethyl methacrylate | 5% |
| 1,3-butylene glycol | 6% |
| Phenoxyethanol | 0.5% |
| Hydrophobized pigment- | 6% |
| grade titanium oxide | |
| Hydrophobized yellow iron oxide | 1.2% |
| Hydrophobized red iron oxide | 0.4% |
| Hydrophobized black iron oxide | 0.1% |
| Ion-exchanged water | Balance |

### Example 3: Sunscreen

| | |
|---|---|
| Dodecamethylcylohexasiloxane | 10.5% |
| Polydimethylsiloxane (6CS) | 10.5% |
| Octyl methoxycinnamate | 6% |
| Octocrylene | 2% |
| Diethylaminohydroxybenzoyl hexyl benzoate | 1.5% |
| Rice bran wax | 3% |
| Sorbitan sesquiisostearate | 2% |
| Polyoxyethylene/methylpolysiloxane copolymer | 2% |
| Polymethyl methacrylate | 5% |
| 1,3-butylene glycol | 6% |
| Phenoxyethanol | 0.5% |
| Hydrophobized fine-particle titanium oxide | 8% |
| Hydrophobized fine-particle zinc oxide | 5% |
| Ion-exchanged water | Balance |

### Example 4: Sunscreen for Body

| | |
|---|---|
| Dodecamethylcylohexasiloxane | 11% |
| Polydimethylsiloxane (6CS) | 11% |
| Octyl methoxycinnamate | 5% |
| Diethylaminohydroxybenzoyl hexyl benzoate | 0.5% |
| Bis-ethylhexyloxyphenol methoxyphenyl triazine | 0.5% |
| Homosalate | 2% |
| 4-tert-butyl-4′-methoxydibenzoylmethane | 1.5% |
| Rice bran wax | 2.5% |
| Sorbitan sesquiisostearate | 2% |
| Polyoxyethylene/methylpolysiloxane copolymer | 2% |
| Polymethyl methacrylate | 5% |
| 1,3-butylene glycol | 6% |
| Phenoxyethanol | 0.5% |
| Hydrophobized fine-particle titanium oxide | 6% |
| Hydrophobized fine-particle zinc oxide | 8% |
| Phenylbenzimidazole sulfonic acid | 3% |
| Triethanolamine | 2.3% |
| Ion-exchanged water | Balance |

### Example 5: Foundation (Powdery-Finish Type)

| | |
|---|---|
| Dodecamethylcylohexasiloxane | 15% |
| Polydimethylsiloxane (6CS) | 15% |
| Octyl methoxycinnamate | 7% |
| Rice bran wax | 2.5% |
| Paraffin wax | 1% |
| Palmitic acid | 0.15% |
| Distearyldimonium chloride | 0.2% |
| Sorbitan sesquiisostearate | 2% |
| PEG-10 dimethicone | 2% |
| Hydrophobized pigment-grade titanium oxide | 6% |
| Hydrophobized red iron oxide | 0.4% |
| Hydrophobized yellow iron oxide | 2% |
| Hydrophobized black iron oxide | 0.05% |
| Hydrophobized fine-particle titanium oxide | 5% |
| Polymethylsilsesquioxane | 5% |
| Methyl methacrylate crosspolymer | 1% |
| Nylon-12 | 1% |
| Synthesized phlogopite | 1% |
| Barium sulfate | 1% |
| Glycerin | 1% |
| 1,3-propanediol | 5% |
| Phenoxyethanol | 0.5% |
| Ion-exchanged water | Balance |

### Example 6: Foundation (Powdery-Finish Type)

| | |
|---|---|
| Dodecamethylcylohexasiloxane | 15% |
| Polydimethylsiloxane (6CS) | 15% |
| Octyl methoxycinnamate | 7% |
| Rice bran wax | 2.5% |
| Paraffin wax | 1% |
| Palmitic acid | 0.15% |
| Distearyldimonium chloride | 0.2% |
| Sorbitan sesquiisostearate | 2% |
| Dimethicone/polyglycerin-3 crosspolymer | 1% |
| Dimethicone/PEG-10/15 crosspolymer | 1% |
| Hydrophobized pigment-grade titanium oxide | 6% |
| Hydrophobized red iron oxide | 0.4% |
| Hydrophobized yellow iron oxide | 2% |
| Hydrophobized black iron oxide | 0.05% |
| Hydrophobized fine-particle titanium oxide | 5% |
| Vinyl dimethicone/methicone silsesquioxane crosspolymer | 1% |
| Silica | 1% |
| Synthesized phlogopite | 1% |
| Barium sulfate | 1% |
| Glycerin | 1% |
| DPG | 5% |
| Phenoxyethanol | 0.5% |
| Ion-exchanged water | Balance |

### Example 7: Foundation (Powdery-Finish Type)

| | |
|---|---|
| Dodecamethylcylohexasiloxane | 15% |
| Polydimethylsiloxane (6CS) | 15% |
| Octyl methoxycinnamate | 5% |
| Rice bran wax | 2.5% |
| Paraffin wax | 1% |
| Palmitic acid | 0.15% |
| Distearyldimonium chloride | 0.2% |
| Sorbitan sesquiisostearate | 2% |
| PEG-10 dimethicone | 2% |
| Hydrophobized pigment-grade titanium oxide | 6% |
| Hydrophobized red iron oxide | 0.4% |
| Hydrophobized yellow iron oxide | 2% |
| Hydrophobized black iron oxide | 0.05% |
| Hydrophobized fine-particle titanium oxide | 5% |
| Polymethylsi lsesquioxane | 5% |
| Nylon-12 | 3% |
| Synthesized phlogopite | 1% |
| Barium sulfate | 1% |
| Glycerin | 1% |
| DPG | 5% |
| Phenoxyethanol | 0.5% |
| Ion-exchanged water | Balance |

1.-10. (canceled)

11. A water-in-oil solid cosmetic, comprising:
a continuous-phase oil that further comprises an organic modified silicone and forms a continuous oil-phase;
a wax that is compatibly soluble in said continuous-phase oil;
an ultraviolet light absorber;
an aqueous-phase; and
wherein said water-in-oil solid cosmetic is solidified with said wax;
wherein a plurality of oil-phase droplets and a plurality of aqueous phase-droplets respectively disperse in separate in said continuous-phase oil dissolving said wax; and
wherein said plurality of oil-phase droplets comprise said ultraviolet light absorber.

12. The water-in-oil solid cosmetic, according to claim 11, wherein:
said aqueous-phase is 5 to 50% by mass in a total mass of said water-in-oil solid cosmetic.

13. The water-in-oil solid cosmetic, according to claim 11, wherein:
said organic modified silicone further comprises:
at least 50% by mass of a linear-chain silicone oil in said continuous-phase oil.

14. The water-in-oil solid cosmetic, according to claim 11, wherein:
said ultraviolet light absorber in said continuous oil-phase is 3 to 40% by mass in the total mass of said water-in-oil solid cosmetic.

15. The water-in-oil solid cosmetic, according to claim 11, wherein:
said continuous oil-phase further comprises:
at least one oil component selected from a group consisting of an ester oil that is a liquid at 25° C. and 1 atmospheric pressure;
said organic modified silicone having a benzene ring in a chemical structure thereof; and
a hydrocarbon oil having a boiling point of not-higher than 300° C. at 1 atmospheric pressure, wherein said respective oil components are at most 30% by mass in said continuous oil-phase.

16. A water-in-oil solid cosmetic, comprising:
a continuous-phase oil that further comprises an organic modified silicone and forms a continuous oil-phase;
a wax that is compatibly soluble in said continuous-phase oil;
an ultraviolet light absorber;
an aqueous-phase; and
wherein said water-in-oil solid cosmetic is solidified with said wax; and
wherein said ultraviolet light absorber is 3 to 40% by mass in a total mass of said water-in-oil solid cosmetic.

17. The water-in-oil solid cosmetic according to claim 16, wherein:
said aqueous-phase is 5 to 50% by mass in the total mass of said water-in-oil solid cosmetic.

18. The water-in-oil solid cosmetic according to claim 16, wherein:
said organic modified silicone further comprises:
at least 50% by mass of a linear-chain silicone oil in said continuous-phase oil.

19. The water-in-oil solid cosmetic according to claim 16, wherein:
said continuous oil-phase further comprises:
at least one oil component selected from a group consisting of an ester oil that is a liquid at 25° C. and 1 atmospheric pressure;
said organic modified silicone having a benzene ring in a chemical structure thereof; and
a hydrocarbon oil having a boiling point of not-higher than 300° C. at 1 atmospheric pressure, wherein said respective oil components are at most 30% by mass in said continuous oil-phase.

20. The water-in-oil solid cosmetic, according to claim 16, wherein:
a plurality of oil-phase droplets and a plurality of aqueous-phase droplets respectively disperse in separate in said continuous-phase oil dissolving said wax, and said oil-phase droplets further comprise said ultraviolet light absorber.
